(19) United States
(12) Patent Application Publication
(10) Pub. No.: US 2012/0238815 A1
(43) Pub. Date: Sep. 20, 2012

(54) INSERTION AID FOR ENDOSCOPE

(75) Inventors: Shuji KOMI, Ashigarakami-gun (JP); Masaya Inoue, Ashigarakami-gun (JP); Masayuki Iwasaka, Ashigarakami-gun (JP)

(73) Assignee: Fujifilm Corporation, Tokyo (JP)

(21) Appl. No.: 13/420,486

(22) Filed: Mar. 14, 2012

(30) Foreign Application Priority Data
Mar. 17, 2011 (JP) JP 2011-059200

(51) Int. Cl. A61B 1/00

(52) U.S. Cl. 600/114

(57) ABSTRACT

An insertion aid for an endoscope includes a tubular body having a passage through which the insertion part is passed; and a sidewall opening part disposed in a distal portion of a sidewall portion of the tubular body, a distal end of the insertion part passed through the passage being capable of being fed from the sidewall opening part, wherein the sidewall opening part is composed of a slot whose longitudinal direction corresponds to an axial direction of the tubular body, and the sidewall opening part is provided with a tapered portion having an opening width gradually decreasing in the longitudinal direction, and is configured to satisfy the following inequality relationship: Wmin<D<Wmax, where Wmax is a maximum opening width of the tapered portion, Wmin is a minimum opening width thereof, and D is an outer diameter of the insertion part.

### BACKGROUND OF THE INVENTION

1. Field of the Invention

The present invention relates to an insertion aid for an endoscope, and in particular to an insertion aid for an endoscope used in inserting an insertion part of an endoscope into a thin lumen such as the biliary tract or the pancreatic duct.

2. Description of the Related Art

In recent years, endoscopic examination or treatment of pancreaticobiliary diseases, such as biliary tract cancer, pancreas cancer, cholelithiasis, and choledocholithiasis, has been widely spreading in the field of medicine. These endoscopic examination and treatment have the advantages of being less invasive and imposing less strain on patients than conventional surgical treatment.

As a method for these examination and treatment, for example, there is ERCP (Endoscopic Retrograde Cholangio-Pancreatography). The ERCP is a diagnosis method using an endoscope to inject contrast medium into the biliary tract or the pancreatic duct and photograph the injected region with a fluoroscope. In the method of injecting the contrast medium, first, an insertion part of the endoscope is inserted into the duodenum. Then, a cannula (thin tube) is fed from a forceps exit of the insertion part, the cannula is inserted from the major duodenal papilla selectively into the biliary tract or the pancreatic duct, a contrast medium is injected into the biliary tract or the pancreatic duct through the cannula, and the injected portion is photographed by a fluoroscope.

A method of confirming the presence or absence of a site of stenosis inside the biliary tract or the pancreatic duct, sampling and examining cells or tissues (cytodiagnosis, biopsy), removing calculi, or the like is also known to be performed by inserting the insertion part of a thin endoscope, generally called cholangioscope or pancreatoscope, into the biliary tract or the pancreatic duct.

Under these circumstances, when the insertion part of the endoscope is inserted into a body cavity, an insertion aid for an endoscope (also called “overtube” or “sliding tube”) is used together. As one example of the insertion aid for an endoscope is disclosed in Japanese Patent Application Laid-Open No. S60-185532, this insertion aid for an endoscope includes a tubular body used as a guide through which the insertion part of the endoscope is passed, a distal sidewall portion of the tubular body being provided with an opening part (referred to as sidewall opening part below) from which a distal end of the insertion part can be fed. According to this insertion aid for an endoscope, when the insertion part of the endoscope is inserted into a body cavity, it is made possible by inserting the insertion part covered with the tubular body to perform easy insertion of the insertion part while preventing undesired bending or flexing of the insertion part. Further, by feeding the distal end of the insertion part from the sidewall opening part of the tubular body and guiding the same into a body cavity (for example, the biliary duct), the insertion part can be inserted further into a deep portion of the body cavity.

Further, Japanese Patent Application Laid-Open No. S62-22623 discloses an insertion aid for an endoscope, which is provided with an inflatable and deflatable balloon positioned more distally than the sidewall opening part of the tubular body. According to this insertion aid for an endoscope, after the insertion part covered with the tubular body in the manner described above is inserted to a desired position in the body cavity, the sidewall opening part can be held at the desired position (for example, at a position opposite to the major duodenal papilla) by inflating the balloon so as to come into close contact with an inner wall of the body cavity. This makes it possible to insert the insertion part further stably into a deep portion of the body cavity.

### SUMMARY OF THE INVENTION

In such conventional insertion aids for an endoscope as disclosed in Japanese Patent Application Laid-Open Nos. S60-185532 and S62-22623, however, since the sidewall opening part is composed of a slot whose longitudinal direction corresponds to an axial direction of the tubular body, and the slot is configured to have a width unchanged in the longitudinal direction, the insertion part fed from the sidewall opening part tends to be displaced, and therefore the insertion part cannot be inserted stably into a deep portion of the body cavity.

The present invention has been made in view of these circumstances, and an object of the present invention is to provide an insertion aid for an endoscope that makes it possible to perform smooth and easy insertion of the insertion part while preventing displacement of the insertion part of the endoscope fed from the sidewall opening part of the tubular body.

In order to achieve the above object, an insertion aid for an endoscope according to a first aspect of the present invention is an insertion aid for an endoscope through which an insertion part of an endoscope is passed to guide the endoscope, the insertion aid including a tubular body having a passage through which the insertion part is passed; and a sidewall opening part disposed in a distal portion of a sidewall portion of the tubular body, a distal end of the insertion part passed through the passage being capable of being fed from the sidewall opening part, wherein the sidewall opening part is composed of a slot whose longitudinal direction corresponds to an axial direction of the tubular body, and the sidewall opening part is provided with a tapered portion having an opening width gradually decreasing in the longitudinal direction, and is configured to satisfy the following inequality relationship:

Wmin<D<Wmax,

where Wmax is a maximum opening width of the tapered portion, Wmin is a minimum opening width thereof, and D is an outer diameter of the insertion part.

According to the first aspect, the insertion part fed from the sidewall opening part abuts on the tapered portion so that the insertion part is prevented from being displaced. This makes it possible to guide the insertion part in a predetermined direction, thereby stably inserting the insertion part smoothly and easily into a deep portion of the body cavity.

An insertion aid for an endoscope according to a second aspect of the present invention is the insertion aid for an endoscope according to the first aspect, wherein the opening width of the tapered portion of the sidewall opening part gradually decreases in the longitudinal direction toward a proximal end of the tubular body.

The second aspect of the present invention is a preferred aspect in a case where the bending angle of the insertion part fed from the sidewall opening part is relatively large, and a lateral surface of the insertion part abuts on the tapered portion so that the insertion part is prevented from being displaced. This makes it possible to guide the insertion part in a predetermined direction, thereby stably inserting the insertion part smoothly and easily into a deep portion of the body cavity.

An insertion aid for an endoscope according to a third aspect of the present invention is the insertion aid for an endoscope according to the first aspect, wherein the opening width of the tapered portion gradually decreases in the longitudinal direction toward a distal end of the tubular body.

The third aspect of the present invention is a preferred aspect in a case where the bending angle of the insertion part fed from the sidewall opening part is relatively small, and a lateral surface of the insertion part abuts on the tapered portion so that the insertion part is prevented from being displaced. This makes it possible to guide the insertion part in a predetermined direction, thereby stably inserting the insertion part smoothly and easily into a deep portion of the body cavity.

An insertion aid for an endoscope according to a fourth aspect of the present invention is the insertion aid for an endoscope according to the first aspect, wherein the tapered portion of the sidewall opening part is composed of a first tapered portion having an opening width gradually decreasing in the longitudinal direction toward a proximal end of the tubular body, and a second tapered portion having an opening width gradually decreasing in the longitudinal direction toward a distal end of the tubular body.

In the fourth aspect of the present invention, since the sidewall opening part is provided with two tapered portions (a first tapered portion and a second tapered portion) different in shape, it becomes possible to select which tapered portion the insertion part abuts on, according to the type or the operational content (the bending angle, or the like) of the insertion part, resulting in improvement in the operability of the insertion part.

An insertion aid for an endoscope according to a fifth aspect of the present invention is the insertion aid for an endoscope according to the first aspect, wherein the sidewall opening part is composed of a first sidewall opening part including a first tapered portion having an opening width gradually decreasing in the longitudinal direction toward a proximal end of the tubular body, and a second sidewall opening part including a second tapered portion having an opening width gradually decreasing in the longitudinal direction toward a distal end of the tubular body.

In the fifth aspect of the present invention, since the tubular body is provided with two sidewall opening parts (a first sidewall opening part and a second sidewall opening part) different in shape, it becomes possible to select which sidewall opening part which the insertion part is fed from, according to the type or the operational content (the bending angle, or the like) of the insertion part, resulting in improvement in the operability of the insertion part.

According to the present invention, the insertion part fed from the sidewall opening part abuts on the tapered portion so that the insertion part is prevented from being displaced. This makes it possible to guide the insertion part in a predetermined direction, thereby stably inserting the insertion part smoothly and easily into a deep portion of the body cavity.

### BRIEF DESCRIPTION OF THE DRAWINGS

FIG. 1 is an external view showing an endoscopic device to which an insertion aid according to an embodiment of the present invention is applied;

FIG. 2 is a perspective view showing a distal end portion of an insertion part of the endoscope;

FIGS. 3A to 3C are schematic views showing a configuration example of the vicinity of a distal end of the insertion aid according to a first embodiment;

FIG. 4 is a longitudinal sectional view showing the insertion aid shown in FIGS. 3A to 3C with a balloon inflated;

FIG. 5 is an illustration showing a procedure for inserting the insertion part of the endoscope into the biliary tract through the use of the insertion aid;

FIG. 6 is an illustration showing a procedure for inserting the insertion part of the endoscope into the biliary tract through the use of the insertion aid:

FIG. 7 is an illustration showing a procedure for inserting the insertion part of the endoscope into the biliary tract through the use of the insertion aid;

FIG. 8 is a schematic view showing a modification of the insertion aid according to the first embodiment;

FIGS. 9A to 9C are schematic views showing a configuration example of the vicinity of a distal end of the insertion aid according to a second embodiment;

FIG. 10 is a schematic view showing a modification of the insertion aid according to the second embodiment;

FIG. 11 is a schematic view showing a configuration example of the vicinity of a distal end of the insertion aid according to a third embodiment;

FIG. 12 is a schematic view showing a modification of the insertion aid according to the third embodiment;

FIGS. 13A to 13C are schematic views showing a configuration example of the vicinity of a distal end of the insertion aid according to a fourth embodiment;

FIGS. 14A to 14C are schematic views showing a configuration example of the vicinity of a distal end of the insertion aid according to another invention; and

FIGS. 15A and 15B are schematic views showing a modification of the insertion aid according to another invention.

### DETAILED DESCRIPTION OF THE PREFERRED EMBODIMENTS

Preferred embodiments of the present invention will be described in detail below with reference to the accompanying drawings.

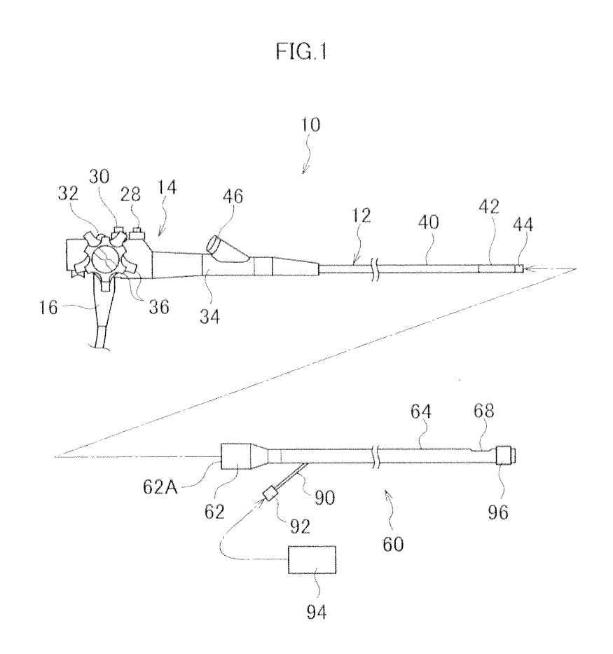

FIG. 1 is an external view showing an endoscopic device to which an insertion aid for an endoscope (simply referred to as “insertion aid” below) according to an embodiment of the present invention is applied. As shown in FIG. 1, an endoscopic device is mainly composed of an endoscope 10 and an insertion aid 60.

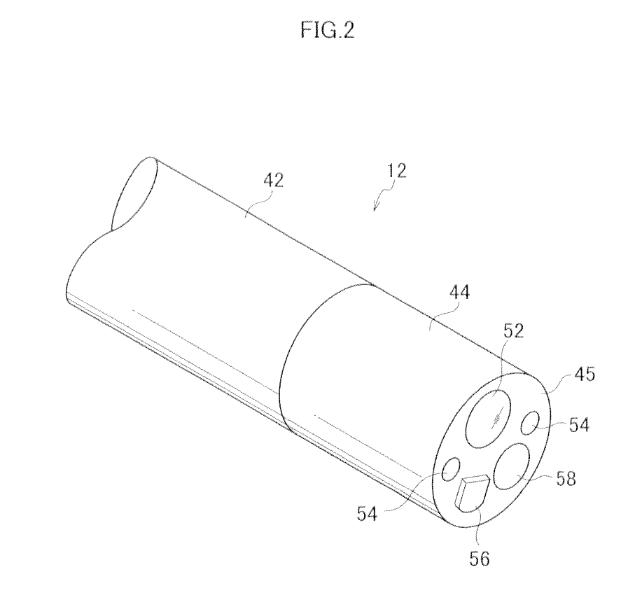

The endoscope 10 is provided with a hand operation part 14, and an insertion part 12 connected to this hand operation part 14 and intended to be inserted into the body. The hand operation part 14 is connected with a universal cable 16, and a distal end of the universal cable 16 is provided with a light guide (LG) connector not shown in the figures. The LG connector is attachably and detachably coupled with a light source device not shown in the figures by which illumination light is transmitted to an illumination optical system 54 (see FIG. 2) described later. The LG connector is also connected with an electrical connector, and this electrical connector is attachably and detachably coupled with a processor that performs image signal processing or the like.

The hand operation part 14 is provided with an air-supply/water-supply button 28, a suction button 30, and a shutter button 32 positioned side by side, and also provided with a pair of angle knobs 36 and 36.

The insertion part 12 is composed of a flexible part 40, a bending part 42, and a distal end portion 44 in this order from the hand operation part 14, and the bending part 42 is bent remotely by rotating the angle knobs 36, 36 of the hand operation part 14. This makes it possible to turn the distal end portion 44 to a desired direction.

As shown in FIG. 2, a distal end face 45 of the distal end portion 44 is provided with an observation optical system 52, the illumination optical systems 54, 54, an air-supply/water-supply nozzle 56, and a forceps exit 58. A CCD (not shown) is disposed behind the observation optical system 52, and a substrate supporting this CCD is connected with a signal cable (not shown). The signal cable is extended to the electrical connector through the insertion part 12, the hand operation part 14, the universal cable 16, and the like to be connected to the processor. Therefore, an observed subject captured by the observation optical system 52 is imaged on a light-receiving surface of the CCD and converted into an electrical signal, and then this electrical signal is outputted to the processor through the signal cable, and converted into a video signal. Accordingly, an observation image is displayed on a monitor connected with the processor.

A light emission end of the light guide (not shown) is disposed behind the illumination optical systems 54, 54 in FIG. 2. This light guide is passed through the insertion part 12, the hand operation part 14, and the universal cable 16 shown in FIG. 1, and an incident end of the light guide is disposed within the LG connector. Therefore, by coupling the LG connector with the light source device, illumination light emitted from the light source device is transmitted to the illumination optical systems 54, 54 through the light guide, and emitted forward from the illumination optical systems 54, 54.

The air-supply/water-supply nozzle 56 in FIG. 2 is in communication with a valve (not shown) operated with the air-supply/water-supply button 28 in FIG. 1, and the valve is further in communication with an air-supply/water-supply connector (not shown) provided to the LG connector. An air-supply/water-supply device shown) is connected with the air-supply/water-supply connector to supply air and water. Therefore, by operating the air-supply/water-supply button 28, air or water can be sprayed from the air-supply/water-supply nozzle 56 to the observation optical system 52.

The forceps exit 58 in FIG. 2 is in communication with a forceps entrance 46 in FIG. 1 via a forceps channel (not shown). Therefore, by inserting a treatment instrument, such as forceps, through the forceps entrance 46, this treatment instrument can be fed from the forceps exit 58. The forceps exit 58 is in communication with a valve (not shown) operated by the suction button 30, and this valve is further connected to a suction connector (not shown) of the LG connector. Therefore, a suction device not shown in the figures is connected with the suction connector, and the valve is operated with the suction button 30 so that a site of lesion or the like can be sucked through the forceps exit 58.

On the other hand, the insertion aid 60 shown in FIG. 1 is provided with a grasping part 62 and a tube main body 64. The tube main body 64 has a tubular shape, and has an inner diameter larger than an outer diameter of the insertion part 12 so that the insertion part 12 of the endoscope 10 can be passed through the tube main body 64. The tube main body 64 is a flexible molded object made of urethane resin, an outer peripheral surface of the tube main body 64 is coated with lubricant, and an inner peripheral surface thereof is also coated with lubricant. The grasping part 62 shown in FIG. 1, which is hard, is water-tightly fitted on the tube main body 64 such that the grasping part 62 is attachably and detachably coupled with the tube main body 64. It should be noted that the insertion part 12 is inserted into the tube main body 64 through a proximal opening part 62A of the grasping part 62.

Here, the configuration of the insertion aid 60 will be described in detail.

### First Embodiment

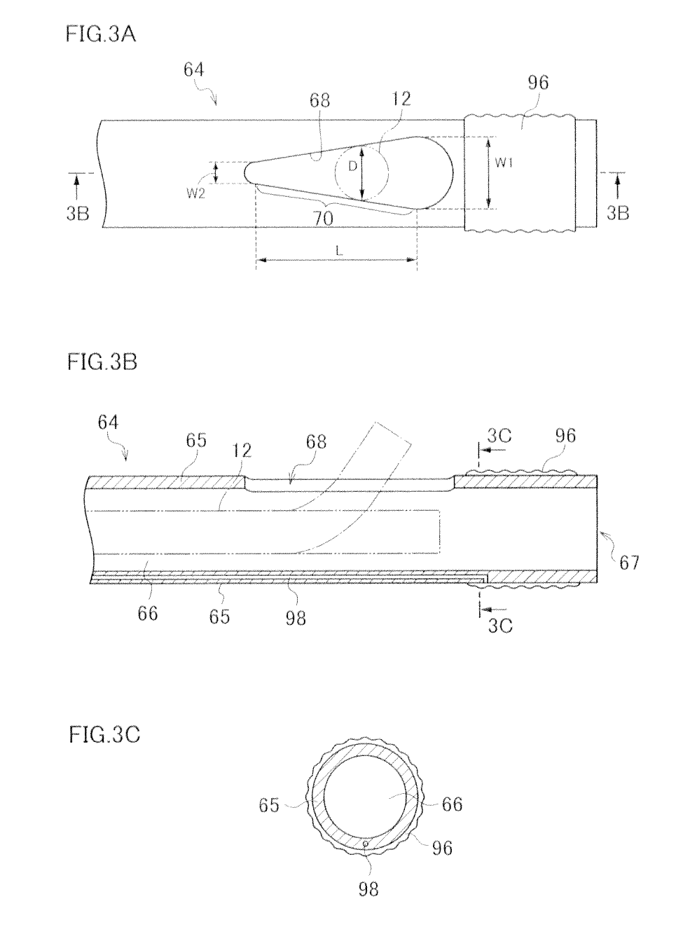

FIGS. 3A to 3C are schematic views showing a configuration example of the vicinity of a distal end of the insertion aid 60 (tube main body 64) according to a first embodiment, FIG. 3A is a plan view of the configuration example, FIG. 3B is a longitudinal sectional view (sectional view taken along line 3B-3B in FIG. 3A) thereof, and FIG. 3C is a cross sectional view (sectional view taken along line 3C-3C in FIG. 3B) thereof.

As shown in FIGS. 3A to 3C, an opening part (referred to as “distal end opening part” below) 67 is formed in a distal end of the tube main body 64, and in the tube main body 64, a passage 66 communicating with the distal end opening part 67 is extended in an axial direction of the tube main body 66. The passage 66 is a channel through which the insertion part 12 of the endoscope 10 (see FIG. 1) is passed, and is substantially circular in cross section perpendicular to the axial direction.

A distal sidewall portion 65 of the tube main body 64 is provided with an opening part (referred to as “sidewall opening part”) 68 for feeding the distal end of the insertion part 12 inserted into the passage 66. This sidewall opening part 68 is composed of a slot (through-hole) whose longitudinal direction corresponds to the axial direction of the tube main body 64, and the sidewall opening part 68 is configured to have a distal end portion which is sufficiently wider than the outer diameter of the insertion part 12 so as to allow the distal end of the insertion part 12 to easily pass through the sidewall opening 68. Though FIGS. 3A to 3C show, as an example of a preferred embodiment, the configuration of the insertion aid 60 in which the distal end portion of the sidewall opening part 68 has an arc shape, it is only necessary for the shape of the sidewall opening part 68 to allow the insertion part 12 to pass through the side wall opening part 68.

The sidewall opening part 68 is also provided with a tapered portion 70 having an opening width (length in a lateral direction of the sidewall opening part 68) gradually decreasing from the distal end portion to a proximal end portion of the sidewall opening part 68. The tapered portion 70 is configured to satisfy the following inequality relationship:

W2<D<W1,

where W1 is a distal opening width (maximum opening width) of the sidewall opening part 68, W2 is a proximal opening width (minimum opening width), and D is the outer diameter of the insertion part 12 of the endoscope 10. This makes it possible for the insertion part 12 fed from the sidewall opening part 68 to abut on a predetermined position in the tapered portion 70 (that is, a position at which the outer diameter D of the insertion part 12 and the opening width of the tapered portion 74 become equal to each other).

The proximal end portion of the sidewall opening part 68 is configured to have an arc shape. It should be noted that the shape of the proximal end portion of the sidewall opening part 68 is not limited to a specific one, and, for example, the proximal end portion may be configured to be an angular shape obtained by extending the tapered portion 70

Further, an inflatable and deflatable balloon 96 is attached to the outer peripheral surface of the widewall portion 65 of the tube main body 64 more distally than the sidewall opening part 68. This balloon 96 is in communication with a fluid channel 98 extended in the axial direction within the sidewall portion 65 of the tube main body 64, and the fluid channel 98 is in communication with a channel in a tube 90 shown in FIG. 1 extended from a proximal end portion of the tube main body 64. A end of the tube 90 is provided with a connector 92, by which the tube 90 is connected with a balloon controller 94.

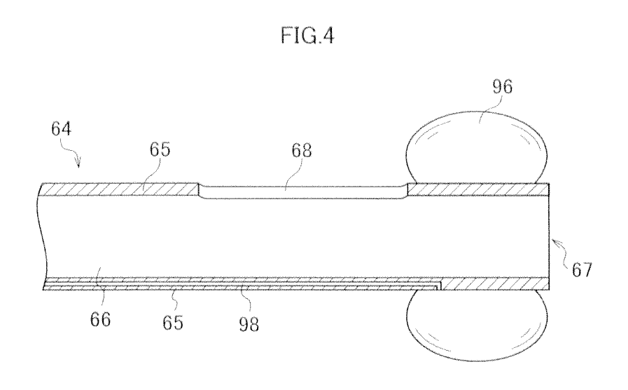

The balloon controller 94 is a device that supplies a fluid, such as air, to the channel in the tube 90 connected by the connector 92, and that also sucks the fluid from the channel in the tube 90. The balloon controller 94 supplies the fluid to the channel in the tube 90, thereby injecting the fluid supplied to the channel into the balloon 96 through the fluid channel 98 in the tube main body 64. This causes the balloon 96 to inflate annularly around the tube main body 64, as shown in FIG. 4. On the other hand, the balloon controller 94 sucks the fluid from the channel in the tube 90, thereby causing the balloon 96 to discharge the fluid injected in the balloon 96 into the fluid channel 98 of the tube main body 64 so that the balloon controller 94 sucks the fluid through the fluid channel 98 and the channel in the tube 90. Thus, the balloon 96 is configured to be deflatable.

Next, the method of operating the endoscopic device thus configured will be described with reference to FIGS. 5 to 7. Though here a case where the insertion part 12 of the endoscope 10 is inserted into a biliary tract 104 will be described as an example, the method is also applied to a case where the insertion part 12 is inserted into a pancreatic duct 106.

First, the insertion part 12 of the endoscope 10 is covered with the insertion aid 60 so as to be passed through the passage 66 of the tube main body 64, and in this state the insertion part 12 and the tube main body 64 are inserted through a patient's mouth, and inserted into a duodenum 100 through the stomach. At this time, the distal end of the insertion part 12 is fed from the distal opening part 67 such that an observation direction (direction of observational field of view) of the observation optical system 52 of the insertion part 12 substantially corresponds to the axial direction of the tube main body 64. Further, the balloon 96 is in a deflated state.

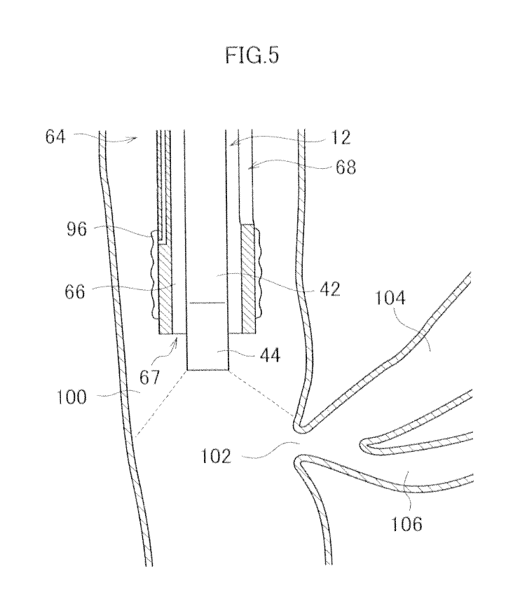

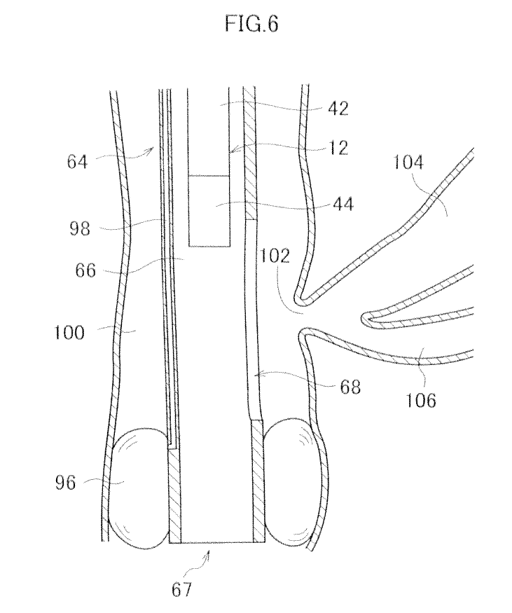

Next, after a major duodenal papilla 102 is confirmed through an observation image observed by the observation optical system 52 of the insertion part 12, the insertion aid 60 is inserted further forward into a deep portion (distal end) of the duodenum 100 so as to position the sidewall opening part 68 of the tube main body 64 to face the major duodenal papilla 102, as shown in FIG. 6. Then, the balloon controller 94 injects the fluid into the balloon 96 to inflate the balloon 96 so as to bring the balloon 96 into close contact with an inner wall of the duodenum 100, thereby fixing the position of the tube main body 64 in the duodenum 100 so as to hold the sidewall opening part 68 of the tube main body 64 in a position opposite to the major duodenal papilla 102.

Next, the insertion part 12 is inserted further forward while being bent, the distal end of the insertion part 12 is fed from the sidewall opening part 68 of the tube main body 64, as shown in FIG. 7, and the distal end is inserted into the biliary tract (common bile duct) 104 through the major duodenal papilla 102.

At this time, since the distal end portion of the sidewall opening part 68 is configured to be sufficiently wider than the outer diameter of the insertion part 12, the distal end of the insertion part 12 can be easily fed from the sidewall opening part 68 by inserting the insertion part 12 to the distal end portion of the sidewall opening part 68 and then bending the insertion part 12. Further, since the sidewall opening part 68 is provided with the tapered portion 70 having an opening width gradually decreasing from the distal end to the proximal end, a lateral surface of the insertion part 12 fed from the sidewall opening part 68 comes to abut on the tapered portion 70 according to gradual increase in the bending angle of the insertion part 12. This causes the insertion part 12 to be guided in a predetermined direction while being prevented by the tapered portion 70 from being displaced in the lateral direction of the sidewall opening part 68, so that the distal end of the insertion part 12 can be easily inserted into the biliary tract 104. Then, the distal end of the insertion part 12 can be inserted further forward into a deep portion of the biliary tract 104.

Thus, according to the first embodiment, the distal end of the insertion part 12 can be easily fed from the distal end portion of the sidewall opening part 68, and displacement of the insertion part 12 can be prevented by making the side surface of the insertion part 12 fed from the sidewall opening part 68 abut on the tapered portion 70. This makes it possible to guide the insertion part 12 in a predetermined direction so that the insertion part 12 can be stably inserted smoothly and easily into a deep portion of the body cavity.

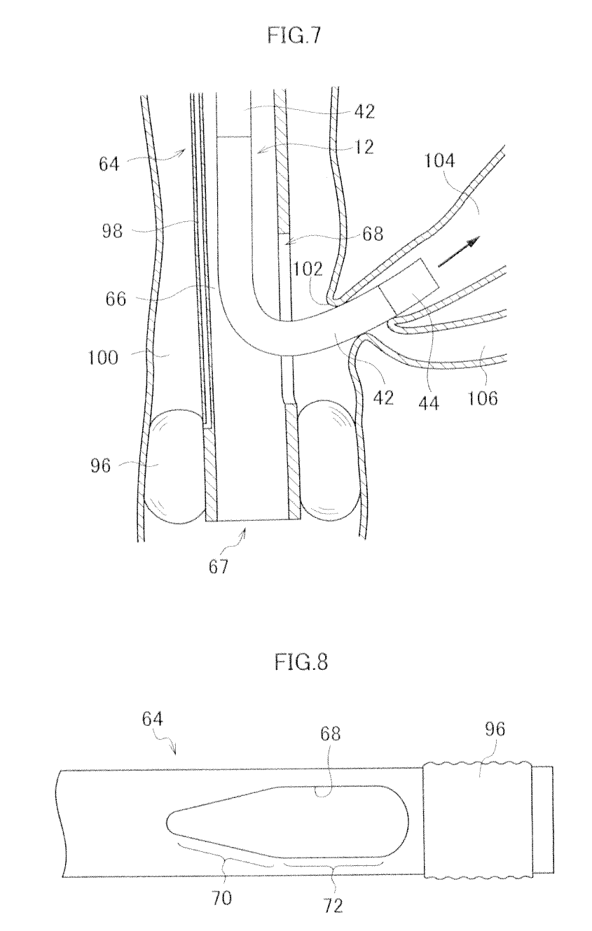

FIG. 8 is a schematic view showing a modification of the insertion aid 60 (tube main body 64) according to the first embodiment. In FIG. 8, components that are identical with or similar to those in FIGS. 3A to 3C are marked with identical reference numerals in order to omit descriptions of the components. In the modification shown in FIG. 8, the sidewall opening part 68 is provided with a tapered portion 70 having an opening width gradually decreasing longitudinally toward the proximal end, and a constant width portion 72 which has a constant opening width and which is positioned on a distal side of the tapered portion 70. According to this modification, it becomes possible to obtain the same advantageous effect as the first embodiment, and it also becomes possible to feed the distal end of the insertion part 12 easily from the constant width portion 72 of the sidewall opening part 68 even if the bending angle of the insertion part 12 is small.

### Second Embodiment

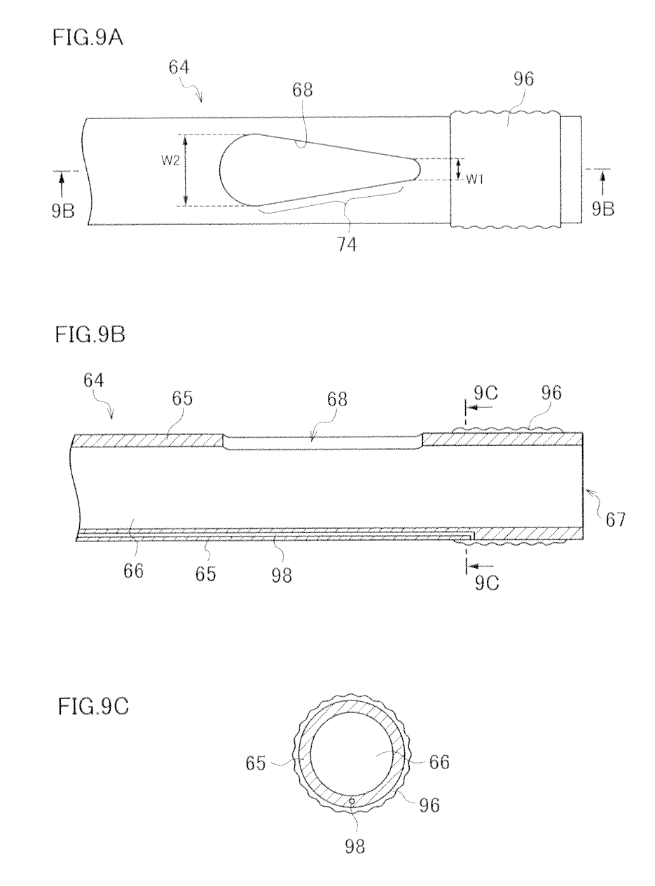

FIGS. 9A to 9C are schematic views showing a configuration example of the vicinity of a distal end of the insertion aid 60 (tube main body 64) according to a second embodiment, FIG. 9A is a plan view of the configuration example, FIG. 9B is a longitudinal sectional view (sectional view taken along line 9B-9B in FIG. 9A) thereof, and FIG. 9C is a cross sectional view (sectional view taken along line 9C-9C in FIG. 9B) thereof. In FIGS. 9A to 9C, components that are identical with or similar to those in FIGS. 3A to 3C are marked with identical reference numerals in order to omit descriptions of the components.

In the second embodiment, the shape of the sidewall opening part 68 is a reversed shape of the sidewall opening part 68 in the first embodiment. That is, as shown in FIGS. 9A to 9C, the sidewall opening part 68 in the second embodiment is different from that in the first embodiment in that the proximal end portion is configured to be sufficiently wider than the outer diameter of the insertion part 12, and in that a tapered portion 74 having an opening width gradually decreasing from the proximal end toward the distal end is provided.

This tapered portion 74 is configured to satisfy the following inequality relationship:

W1<D<W2,

where W1 is a distal opening width (maximum opening width), W2 is a proximal opening width (minimum opening width), and D is the outer diameter of the insertion part 12 of the endoscope 10. This makes it possible for the insertion part 12 fed from the sidewall opening part 68 to abut on a predetermined portion in the tapered portion 74 (that is, a position at which the outer diameter D of the insertion part 12 and the opening width of the tapered portion 74 become equal to each other).

According to the second embodiment, it becomes possible to feed the distal end of the insertion part 12 easily from the proximal end portion of the sidewall opening part 68, and displacement of the insertion part 12 can be prevented by causing the side surface of the insertion part 12 fed from the sidewall opening part 68 to abut on the tapered portion 74. This makes it possible for the insertion part 12 to be guided in a predetermined direction so that the insertion part 12 can be stably inserted smoothly and easily into a deep portion of the body cavity.

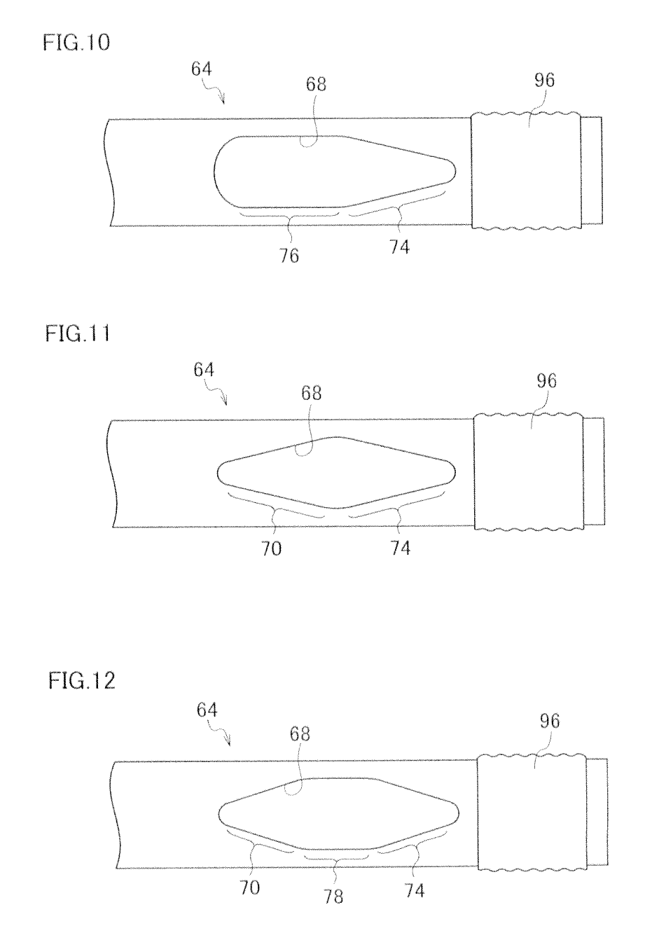

FIG. 10 is a schematic view showing a modification of the insertion aid 60 (tube main body 64) according to the second embodiment. In FIG. 10, components that are identical with or similar to those in FIGS. 9A to 9C are marked with identical reference numerals in order to omit descriptions of the components. In the modification shown in FIG. 10, the sidewall opening part 68 is provided with a tapered portion 74 having an opening width gradually decreasing longitudinally toward the distal end, and a constant width portion 76 which has a constant opening width and which is positioned on a proximal side of the tapered portion 74. According to this modification, it becomes possible to obtain the same advantageous effect as the second embodiment, and it also becomes possible to feed the distal end of the insertion part 12 easily from the constant width portion 76 of the sidewall opening part 68 even if the bending angle of the insertion part 12 is small.

### Third Embodiment

FIG. 11 is a schematic view showing a configuration example of the vicinity of a distal end of the insertion aid 60 (tube main body 64) according to a third embodiment. It should be noted that, in FIG. 11, components that are identical with or similar to those in FIGS. 3A to 3C are marked with identical reference numerals in order to omit descriptions of the components.

In the third embodiment, as shown in FIG. 11, the sidewall opening part 68 has a substantially-diamond shape in planar view, and is configured to be provided with the first tapered portion 70 having an opening width gradually decreasing from a longitudinal central portion of the sidewall opening part 68 toward the proximal end, and the second tapered portion 74 having an opening width gradually decreasing from the central portion toward the distal end.

According to the third embodiment, it is possible to feed the insertion part 12 easily from the central portion having the maximum opening width of the sidewall opening part 68, and it also becomes possible to stably insert the insertion part 12 smoothly and easily into a deep portion of the body cavity while preventing displacement of the insertion part 12 by causing the insertion part 12 fed from the sidewall opening part 68 to abut on the first tapered portion 70 or the second tapered portion 74.

FIG. 12 is a schematic view showing a modification of the insertion aid 60 (tube main body 64) according to the third embodiment. In FIG. 12, components that are identical with or similar to those in FIG. 11 are marked with identical reference numerals in order to omit descriptions of the components. In the modification shown in FIG. 12, the sidewall opening part 68 is provided with a constant width portion 78 having a constant opening width between the first tapered portion 70 and the second tapered portion 74. According to this modification, it becomes possible to obtain the same advantageous effect as the third embodiment, and it also becomes possible to feed the distal end of the insertion part 12 easily from the constant width portion 78 of the sidewall opening part 68 even if the bending angle of the insertion part 12 is small.

### Fourth Embodiment

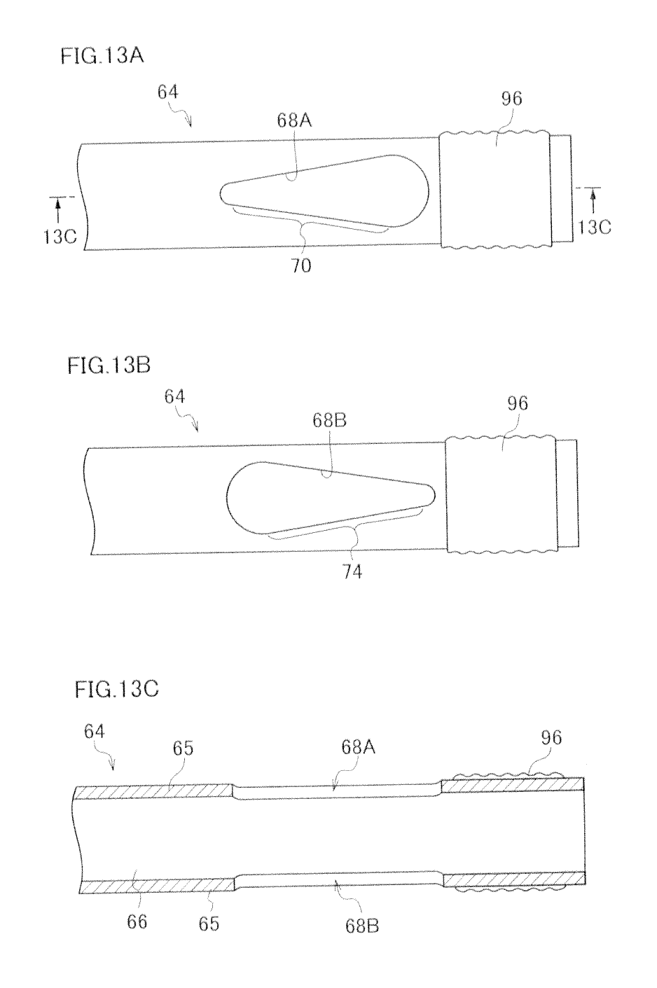

FIGS. 13A to 13C are schematic views showing a configuration example of the vicinity of a distal end of the insertion aid 60 (tube main body 64) according to a fourth embodiment, FIG. 13A is a top view of the configuration example, FIG. 13B is a bottom view thereof, and FIG. 13C is a longitudinal sectional view (sectional view taken along line 13C-13C in FIG. 13A) thereof. It should be noted that, in FIGS. 13A to 13C, components that are identical with or similar to those in FIGS. 3A to 3C and FIGS. 9A to 9C are marked with identical reference numerals in order to omit descriptions of the components.

The fourth embodiment is an aspect of the present invention obtained by combining the first embodiment and the second embodiment. That is, as shown in FIGS. 13A to 13C, a first sidewall opening part 68A is formed in the tube main body 64, and a second sidewall opening part 68B is also formed therein so as to be positioned across the passage 66 from the first sidewall opening part 68A. The first sidewall opening part 68A and the second sidewall opening part 68B are each formed in a slot-like shape whose longitudinal direction corresponds to the axial direction of the tube main body 64. Further, the first sidewall opening part 68A is provided with the first tapered portion 70 having an opening width gradually decreasing from the distal end longitudinally toward the proximal end. On the other hand, the second sidewall opening part 68B is provided with the second tapered portion 74 narrowing gradually from the proximal end longitudinally toward the distal end.

According to the fourth embodiment, it becomes possible to feed the insertion part 12 selectively from the first sidewall opening part 68A or the second sidewall opening part 68B according to the type or the operational content (the bending angle or the like) of the insertion part 12 passed through the passage 66 of the tube main body 64, and therefore the operability of the insertion part 12 is improved.

<Disclosure of Another Invention>

Next, another invention relating to the insertion aid 60 will be described.

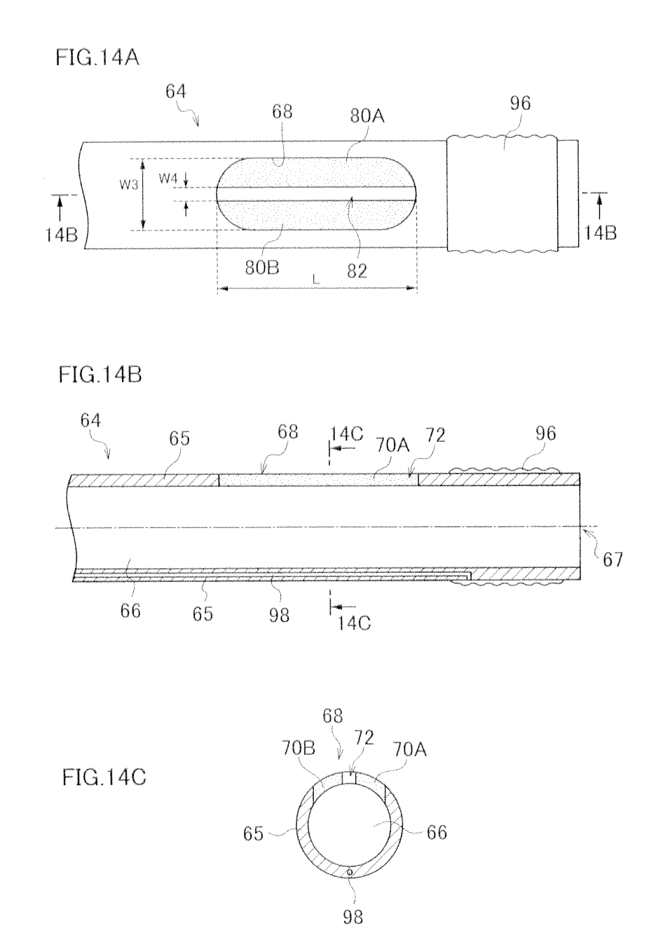

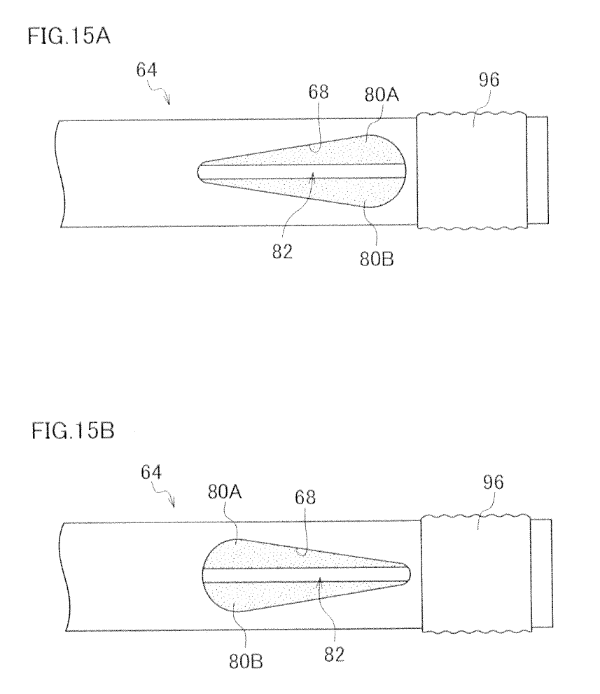

FIGS. 14A to 14C are schematic views showing a configuration example of the vicinity of a distal end of the insertion aid 60 (tube main body 64) according to another embodiment of the present invention, FIG. 14A is a plan view, FIG. 14B is a longitudinal sectional view (sectional view taken along line 14B-14B in FIG. 14A) thereof, and FIG. 14C is a cross sectional view (sectional view taken along line 14C-14C in FIG. 14B) thereof. It should be noted that, in FIGS. 14A to 14C, components that are identical with or similar to those in FIGS. 3A to 3C are marked with identical reference numerals in order to omit descriptions of the components.

In the configuration example of shown in FIGS. 14A to 14C, the sidewall opening part 68 is formed in a slot-like shape having a constant opening width in the longitudinal direction, and elastic films 80A and 80B are disposed inside the sidewall opening part 68. These elastic films 80A and 80B are formed so as to close both side portions in the lateral direction of the sidewall opening part 68, and a slit-like space 82 in the longitudinal direction of the sidewall opening part 68 is formed between the elastic films 80A and 80B. The sidewall opening part 68 is configured to satisfy the following inequality:

W3>D>W4,

where W3 is the opening width of the sidewall opening part 68, W4 is an opening width of the slit-like space, and D is the outer diameter of the insertion part 12. The length of the sidewall opening part 68 (the length of the slit-like space 82) L is configured to be sufficiently larger than the outer diameter D of the insertion part 12, preferably, configured to be equal to or larger than the length of the bending part 42 of the insertion part 12.

The material of the elastic films 80A and 80B is not limited to a specific one, but, in order to secure the field of view for feeding the insertion part 12 from the sidewall opening part 68, it is preferred that the elastic films 80A and 80B are made of a transparent rubber, for example. The transparent rubber is, preferably, a silicone rubber, a urethane rubber, an acrylic rubber, or the like, and among these rubbers a transparent silicone rubber is most preferred.

According to the configuration example shown in FIGS. 14A to 14C, since both sides of the slit-like space 82 formed inside the sidewall opening part 68 are defined by the elastic films 80A and 80B, the elasticity of the elastic films 80A and 80B allows the distal end of the insertion part 12 to be fed from the slit-like space 82 even if the opening width W4 of the slit-like space 82 is smaller than that outer diameter D of the insertion part 12.

Further, the insertion part 12 fed from the slit-like space 82 is held at a central portion in the lateral direction of the sidewall opening part 68 by a biasing force received from the elastic films 80A and 80B, so that the insertion part 12 is prevented from being displaced in the lateral direction of the sidewall opening part 68. This makes it possible to stably insert the insertion part 12 smoothly and easily into a deep portion of a lumen.

FIGS. 14A to 14C illustrate the case where the sidewall opening part 68 has a slot-like shape having a constant opening width, but this is not a limitation, and, for example, as shown in FIG. 5A or FIG. 5B, the sidewall opening part 68 shown in the first embodiment or the second embodiment can be configured to be provided with the elastic films 80A and 80B. According to such a configuration, with the advantageous effect of the first embodiment or the second embodiment, the biasing force from the elastic films 80A and 80B makes it possible to prevent displacement of the insertion part 12 more reliably. It should be noted that the shape of the sidewall opening part 68 to which the elastic films 80A and 80B described above is not limited to a specific one, and, for example, the elastic films 80A and 80B is applicable to the sidewall opening parts 68 shown in FIGS. 8 and 10 to 12.

Hereinabove, the insertion aid for an endoscope of the present invention has been described specifically, but the present invention is not limited to the above embodiments, and obviously can be modified or varied without departing from the scope of the present invention.

What is claimed is:

1. An insertion aid for an endoscope through which an insertion part of an endoscope is passed to guide the endoscope, the insertion aid comprising:
a tubular body having a passage through which the insertion part is passed; and
a sidewall opening part disposed in a distal portion of a sidewall portion of the tubular body, a distal end of the insertion part passed through the passage being capable of being fed from the sidewall opening part, wherein
the sidewall opening part is composed of a slot whose longitudinal direction corresponds to an axial direction of the tubular body, and the sidewall opening part is provided with a tapered portion having an opening width gradually decreasing in the longitudinal direction, and is configured to satisfy the following inequality relationship:
Wmin<D<Wmax,
where Wmax is a maximum opening width of the tapered portion, Wmin is a minimum opening width thereof, and D is an outer diameter of the insertion part.

2. The insertion aid for an endoscope according to claim 1, wherein
the opening width of the tapered portion of the sidewall opening part gradually decreases in the longitudinal direction toward a proximal end of the tubular body.

3. The insertion aid for an endoscope according to claim 1, wherein
the opening width of the tapered portion of the sidewall opening part gradually decreases in the longitudinal direction toward a distal end of the tubular body.

4. The insertion aid for an endoscope according to claim 1, wherein
the tapered portion of the sidewall opening part is composed of a first tapered portion having an opening width gradually decreasing in the longitudinal direction toward a proximal end of the tubular body, and a second tapered portion having an opening width gradually decreasing in the longitudinal direction toward a distal end of the tubular body.

5. The insertion aid for an endoscope according to claim 1, wherein
the sidewall opening part is composed of a first sidewall opening part including a first tapered portion having an opening width gradually decreasing in the longitudinal direction toward a proximal end of the tubular body, and a second sidewall opening part including a second tapered portion having an opening width gradually decreasing in the longitudinal direction toward a distal end of the tubular body.
